(19) United States
(12) Patent Application Publication
(10) Pub. No.: US 2020/0282155 A1
(43) Pub. Date: Sep. 10, 2020

(54) TAMPER-EVIDENT CLOSURE ASSEMBLY

(71) Applicant: NORDSON CORPORATION, WESTLAKE, OH (US)

(72) Inventors: Peter D. Lewis, Loveland, CO (US); Kyle R. Steele, Loveland, CO (US)

(21) Appl. No.: 16/651,432

(22) PCT Filed: Oct. 3, 2018
(86) PCT No.: PCT/US2018/054067

§ 371 Date: Mar. 27, 2020

Related U.S. Application Data
(60) Provisional application No. 62/568,917, filed on Oct. 6, 2017.

(51) Int. Cl. A61M 5/50; A61M 5/32; A61M 39/10

(52) U.S. Cl. CPC A61M 5/5086; A61M 2205/583; A61M 39/1011; A61M 5/3202

(57) ABSTRACT

A tamper-evident closure assembly includes a connector having a receptacle defined by an outer surface of the connector, the receptacle having a depression and a wall adjacent the depression; a cap configured to attach to the connector and to rotate around the connector; a locking mechanism disposed on the cap, the locking mechanism having a locked configuration and an unlocked configuration, the locking mechanism having a tab movable between the locked configuration and the unlocked configuration; and a plug disposed on the tab, wherein when the locking mechanism is in the unlocked configuration, the plug is disposed outside of the receptacle on the connector, and when the locking mechanism is in the locked configuration, the plug is disposed at least partly within the receptacle, such that the cap is prevented from rotating around the connector.

### CROSS REFERENCE TO RELATED APPLICATIONS

This application claims the benefit of U.S. Provisional Patent App. No. 62/568,917, filed Oct. 6, 2017, the disclosure of which is hereby incorporated by reference herein.

### TECHNICAL FIELD

The present disclosure generally relates to tamper detection devices, and, more particularly, to tamper-evident closure assemblies for medical devices.

### BACKGROUND

Many industrial applications require mechanisms that prevent tampering with a particular product. This is especially the case in the medical profession, where it is important for medical staff and patients to be aware of any tampering to a medical device or substance. Existing technology for detecting and preventing tampering is often cumbersome, difficult to use, increases risks of injury to the user, and increase chance of contamination of the patient or the medical environment. Therefore, there is a need for improved systems and devices for detecting tampering with a device in a safe and efficient manner.

### SUMMARY

Devices and methods for tamper-evident closure assemblies are disclosed. In one embodiment, a tamper-evident closure assembly for use with a medical device includes a connector, a cap, and a locking mechanism. The connector has a receptacle defined by an outer surface of the connector. The receptacle has a depression and a wall adjacent the depression. The cap is configured to attach to the connector and to rotate around the connector. The locking mechanism is disposed on the cap and has a locked configuration and an unlocked configuration. The locking mechanism includes a tab movable between the locked configuration and the unlocked configuration and a plug disposed on the tab. When the locking mechanism is in the unlocked configuration, the plug is disposed outside of the receptacle on the connector, and when the locking mechanism is in the locked configuration, the plug is disposed at least partly within the receptacle, such that the cap is prevented from rotating around the connector.

In another embodiment, a system for medicinal delivery includes a medical device configured to receive a medicinal substance and a tamper-evident closure assembly. The closure assembly includes a connector, a cap, and a locking mechanism. The connector has a retaining mechanism configured to releasably affix the connector to the medical device and a receptacle defined by an outer surface of the connector. The receptacle has a depression and a wall adjacent the depression. The cap is configured to attach to the connector and to rotate around the connector. The locking mechanism is disposed on the cap and has a locked configuration and an unlocked configuration. The locking mechanism includes a tab movable between the locked configuration and the unlocked configuration and a plug disposed on the tab. When the locking mechanism is in the unlocked configuration, the plug is disposed outside of the receptacle on the connector, and when the locking mechanism is in the locked configuration, the plug is disposed at least partly within the receptacle, such that the cap is prevented from rotating around the connector.

In yet another embodiment, a method of removing a tamper-evident closure assembly from a medical device includes the steps of moving the closure assembly from an unlocked configuration to a locked configuration and rotating the closure assembly in the locked configuration until the closure assembly is disconnected from the medical device. The closure assembly includes a locking mechanism having a plug and a connector defining a receptacle thereon.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates an isometric view of a closure assembly in accordance with an embodiment;

FIG. 2 illustrates a front perspective view of a connector in accordance with an embodiment;

FIG. 3 illustrates a cross-sectional view of the closure assembly shown in FIG. 1 in an unlocked configuration;

FIG. 4 illustrates a cross-sectional view of the closure assembly shown in FIGS. 1 and 3 in a locked configuration;

FIG. 5 illustrates an isometric view of a portion of a locking mechanism according to an embodiment;

FIG. 6 illustrates an isometric view of a portion of a locking mechanism according to another embodiment;

FIG. 7 illustrates an isometric view of a closure assembly in an unlocked configuration according to another embodiment;

FIG. 8 illustrates an isometric view of the closure assembly shown in FIG. 7 in a locked configuration;

FIG. 9 illustrates a cross-sectional view of the closure assembly shown in FIG. 7;

FIG. 10 illustrates a cross-sectional view of the closure assembly shown in FIG. 8; and

FIG. 11 illustrates an isometric view of a system with a closure assembly according to an embodiment.

### DETAILED DESCRIPTION OF ILLUSTRATIVE EMBODIMENTS

Systems and methods are disclosed for providing tamper-evident closure assemblies for medical devices. The closure assembly includes a connector with which the assembly connects to a medical device and a tamper-evidence mechanism that informs the user of tampering. Once tampering has been identified, the user may take appropriate steps to remedy the problem.

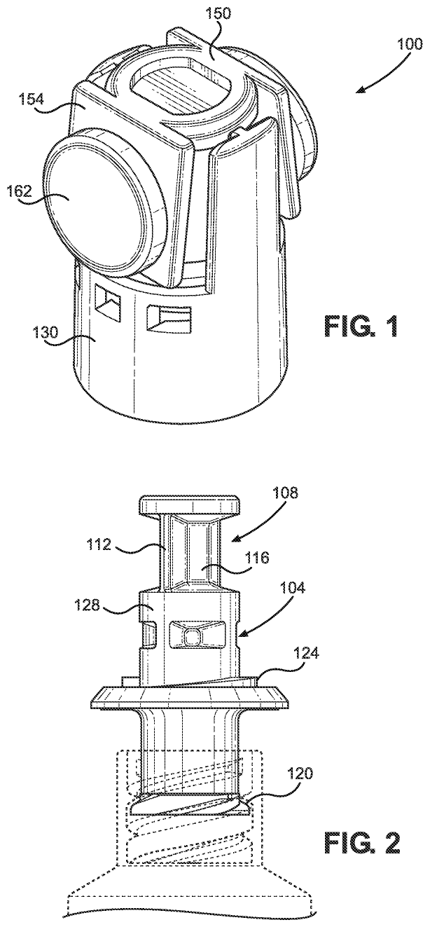

Referring to FIGS. 1-4, a closure assembly 100 includes a connector 104, a cap 130, and a locking mechanism 150. As shown in the illustrative embodiment of FIG. 2, the connector 104 may be substantially cylindrical. However, it will be appreciated that the connector 104 may alternatively comprise a different shape, for example, prismatic, and that the present disclosure is not limited to the shape of the connector 104. The connector 104 may include a retaining mechanism 120 disposed thereon and configured to fixedly attach the connector 104 to a separate component, for example a medical device. The retaining mechanism 120 may be a friction fit connector, a threaded connector, or another suitable connector. In some embodiments, the connector 104 may be configured to attach to standardized medical connectors, for example, Luer lock interfaces.

A receptacle 108 is disposed on the connector 104 and is defined by the outer surface 128 of the connector 104. The receptacle 108 is further defined by a depression 116 and at least one wall 112 that extends from the depression 116 toward the outer surface 128. The receptacle 108 may be defined by multiple walls 112 extending from the depression 116. In some embodiments, the connector 104 includes multiple receptacles 108, for example, 2, 3, 4, or another suitable number of receptacles.

The cap 130 is attached to the connector 104 and is configured to rotate around the centerline of the connector 104 depicted by axis A. It will be understood that the cap 130 may attach to the connector 104 in a variety of ways, for example, via a snap-fitting, and this disclosure is not limited to any specific connection interface between the cap 130 and the connector 104. In some embodiments, the cap 130 may freely rotate around the connector 104 between 0 and 360 degrees, inclusive. In alternative embodiments, the range of rotation may be limited, for example between 0 and 45 degrees, between 0 and 90 degrees, between 0 and 180 degrees, and between 0 and 360 non-inclusive. In some embodiments, the cap 130 may be larger than the connector 104 such that the cap 130 covers the connector 104. Referring to the illustrative embodiment of FIGS. 1-4, the cap 130 encloses the entirety of the connector 104 within itself. In further embodiments, the cap 130 may also enclose at least a portion of a medical device connected to the connector 104 with the retaining mechanism 120. By covering the connection portion of the connector 104, the cap 130 may prevent undesirable access to the retaining mechanism 120. In some embodiments, it may be advantageous to have the cap 130 encompass the connector 104 and the retaining mechanism 120 to prevent tampering with the connector 104 or any components attached to thereto.

The closure assembly 100 further includes a locking mechanism 150 configured to engage with the cap 130, the connector 104, or both. Referring again to FIGS. 1-4, the locking mechanism 150 is disposed on the cap 130 and radially surrounds at least a portion of the cap 130. The locking mechanism 150 can be actuated to lock the rotation of the cap 130 around the connector 104. In an unlocked configuration, the locking mechanism 150 does not obstruct rotation of the cap 130, permitting the cap 130 to rotate around the connector 104 up to its full range of rotation. In a locked configuration, the locking mechanism 150 impedes the full range of rotation of the cap 130. In some embodiments, the locked configuration may permit the cap 130 to rotate at a decreased range of rotation that is smaller than the range of rotation when the device is in the unlocked configuration. In another embodiment, when the locking mechanism 150 is in the locked configuration, the cap 130 is substantially prevented from rotating at all.

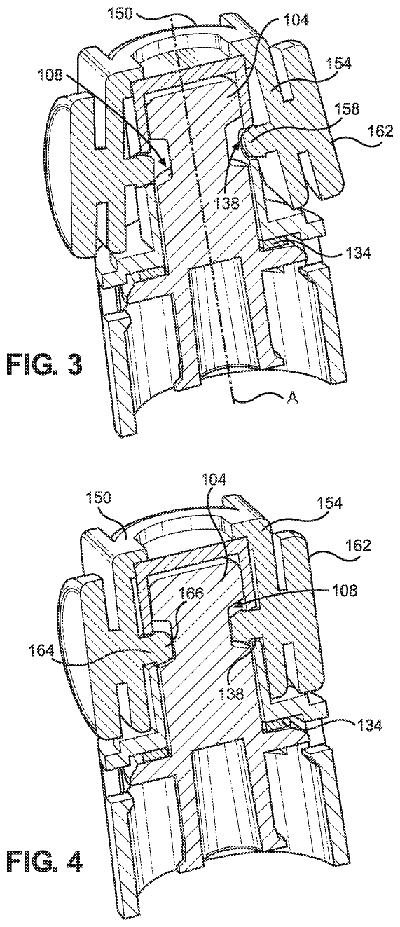

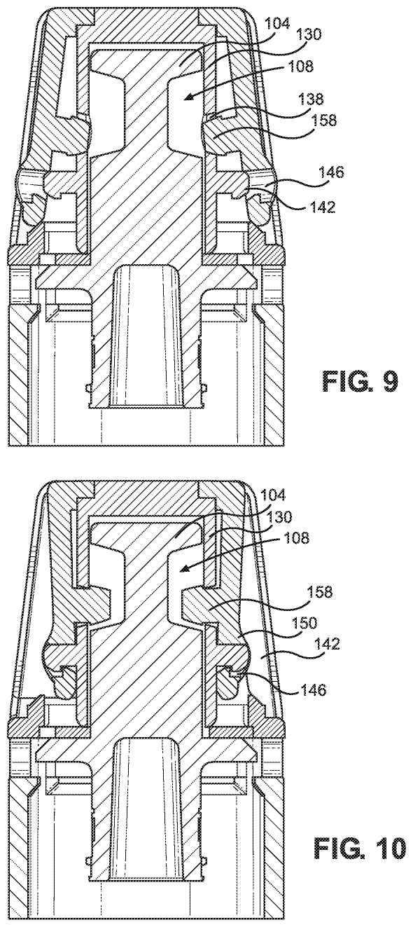

A tab 154 may communicate with the cap 130 and/or the connector 104 to decrease the range of rotation or to completely prevent rotation of the cap 130. The tab 154 may transition between the unlocked configuration where the cap 130 can rotate and the locked configuration where the rotation is limited. Referring to FIGS. 3-4, a plug 158 may be disposed on the tab 154 to interact with the cap 130 and/or the connector 104. The closure assembly 100 may include a plurality of plugs 158, for example 2, 3, 4, or another suitable number. In the illustrative embodiment of FIGS. 1-11, the closure assembly 100 includes two plugs 158.

In some embodiments, when the tab 154 transitions from the unlocked configuration to the locked configuration, the plug 158 engages with a corresponding component of the closure assembly 100. The plug 158 may be dimensioned such that it can pass into the receptacle 108 of the connector 104. As shown in FIG. 3, when the locking mechanism 150 is in the unlocked configuration, the plug 158 is outside the receptacle 108. Referring now to FIG. 4, showing the locking mechanism 150 in the locked configuration, the plug 158 is disposed at least partially within the receptacle 108.

In the locked configuration, the interaction of the plug 158 within the receptacle 108 obstructs rotation of the one or more components. As rotational force is applied to the locking mechanism 150, the cap 130, and/or the connector 104, the plug 158 may contact the wall 112. This contact prevents continued relative rotation between the cap 130 and the connector 104. In some embodiments, the cap 130 may be configured to rotate in multiple directions, for example, clockwise and counterclockwise around the axis A. In such embodiments, the receptacle 108 may include two walls 112 such that, in the locked configuration, the plug 158 may be disposed within the receptacle 108 between the two walls 112. Thus, as rotational force is applied between the cap 130 and the connector 104 in the clockwise direction, the plug 158 contacts one of the two walls 112; conversely, if rotational force is applied in the counterclockwise direction, the plug 158 contacts the other of the two walls 112. Each wall 112 prevents the plug 158 from moving radially out of the receptacle 108. The plug 158, being fixedly connected to the tab 154, thus prevents radial rotation of the locking mechanism 150 around the connector 104.

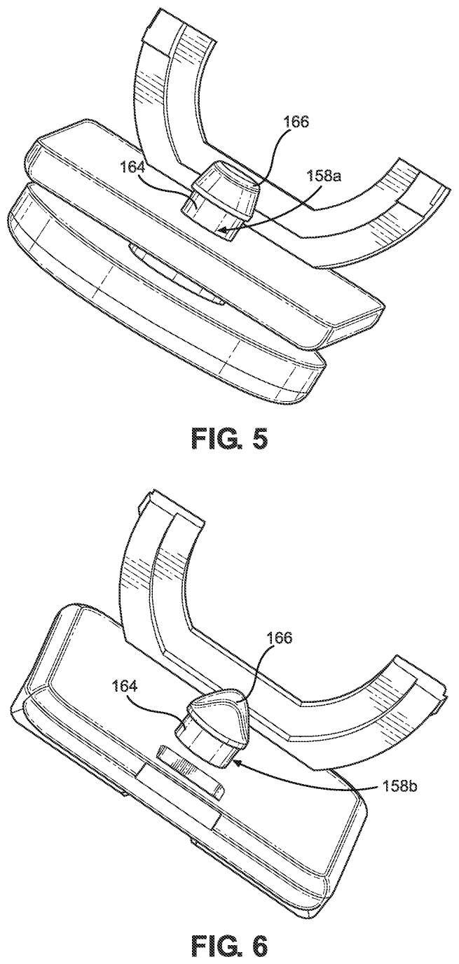

The plug 158 may have a plug body 164 and a plug head 166. The plug body 164 may be substantially cylindrical, or it may have another suitable shape. In some embodiments, the plug head 166 may be wider than the plug body 164. The plug head 166 may be tapered such that the plug head 166 is widest adjacent the plug body 164 and is most narrow at the point farthest away from the plug body 164. Various plug shapes may be used. Referring to FIGS. 5 and 6, two exemplary embodiments of plugs 158 are depicted. Plug 158a shows a plug body 164 that is narrower than the plug head 166. As shown in FIG. 5, the plug head 166 may taper to a flat distal end at the farthest point from the plug body 164. In an alternative embodiment illustrated in FIG. 6, plug 158b may have a plug head 166 that tapers to an angled distal end instead. It will be understood that the plug 158 may have different dimensions, and that this disclosure is not limited to the specific embodiments illustrated in the figures. In some embodiments, the wall 112 may be beveled or sloped in the direction of the depression 116. Thus, wherever the plug 158 contacts the wall 112, the plug 158 may slide along the beveled wall 112 into the receptacle 108. The beveled wall 112 may serve as a guide for the plug 158 to direct it into the receptacle 108.

In some embodiments, the cap 130 may further define an opening 138 extending therethrough. The opening 138 may be adjacent the plug 158. When the locking mechanism 150 moves from the unlocked configuration to the locked configuration, the plug 158 may pass through the opening 138. The opening 138 may be dimensioned such that a plug 158 can pass through it in a first direction toward the connector 104 but not in a second direction opposite the first direction. In some embodiments, the plug 158, having the plug head 166 larger than the plug body 164, may be forced through the opening 138 until a portion of the plug 158 passes through. The plug head 166 may be configured to flex upon contacting the portion of the cap 130 that defines the opening 138, such that the plug head 166 temporarily decreases in size in order to pass through the opening 138. Once the plug head 166 passes through the opening 138, it reverts to its original size. In some embodiments, the plug head 166 is dimensioned with a one-way taper towards the distal end of the plug head 166 that is farthest from the plug body 164. In such embodiments, the plug head 166 may pass through the opening 138 only in one direction towards the connector 104 but is then prevented from passing back through the opening 138 in the opposite direction. This ensures that once the locking mechanism is in the locked configuration and the plug 158 is in the receptacle 108, the plug 158 cannot leave the receptacle 108, and the locking mechanism cannot revert to the unlocked configuration without application of excessive force and/or risking damage to one or more components of the closure assembly 100.

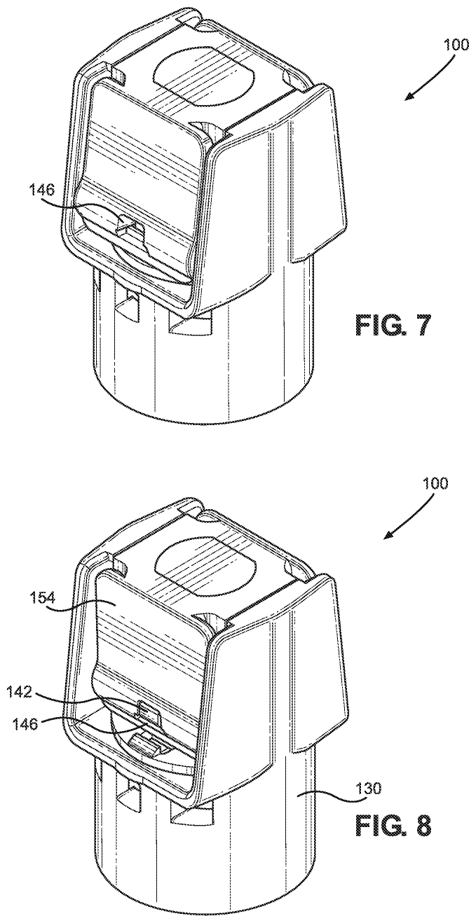

The locking mechanism 150 may further include an actuator 162 that is used to toggle the locking mechanism 150 from the unlocked configuration to the locked configuration. As shown in the illustrative embodiment of FIG. 1, the actuator 162 may be a button. To transition from the unlocked configuration to the locked configuration, a user may apply suitable force to the button in the direction toward the center of the closure assembly 100 (e.g., toward the connector 104). In some embodiments, the suitable force may be a threshold force required to move the plug 158 through the opening 138. Although depicted as a button, it will be understood that the actuator 162 may comprise another structure, for example, a handle, a knob, a switch, or another suitable component that is configured to move the locking mechanism 150 from an unlocked to a locked configuration. In some embodiments, as illustrated in FIGS. 7 and 8, the actuator 162 may be a ridge disposed on the tab 154. In further embodiments, the tab 154 may itself serve as the actuator 162 without an additional structural component.

In some embodiments, the transition from the unlocked configuration to the locked configuration may be designed such that it is irreversible. Once a closure assembly 100 is in the locked configuration, this may be indicative of prior tampering or attempted tampering with the assembly or with a connected component or device. In such embodiments, it may be advantageous to prevent transition from the locked configuration back to the unlocked configuration, as this would result in uncertainty of the existence of previous tampering.

When the locking mechanism 150 is in the locked configuration, it may be further advantageous to notify the user of the transition. In some embodiments, the closure assembly 100 may include an indicator 142 that provides a signal to the user that the assembly is in the locked configuration. When used in the medical field, for example, this signal may alert the user to previous tampering of the closure assembly 100 and/or the medical device associated with it.

Referring to FIGS. 7-10, the indicator 142 may include a protrusion configured to extend from the cap 130. When the locking mechanism 150 is in the unlocked configuration, the indicator 142 is not plainly visible to the user. But, when the locking mechanism 150 is moved into the locked configuration, the indicator 142 becomes visible to notify the user of the transition. In some embodiments, the locking mechanism 150 may include an indicator channel 146 that is dimensioned to receive the indicator 142. As shown in FIG. 7, when the locking mechanism 150 is in the unlocked configuration, the indicator channel 146 is empty and does not house the indicator 142. Referring now to FIG. 8, when the locking mechanism 150 is in the locked configuration, the indicator 142 is disposed at least partly within the indicator channel 146, such that it is plainly visible to the user external the closure assembly 100.

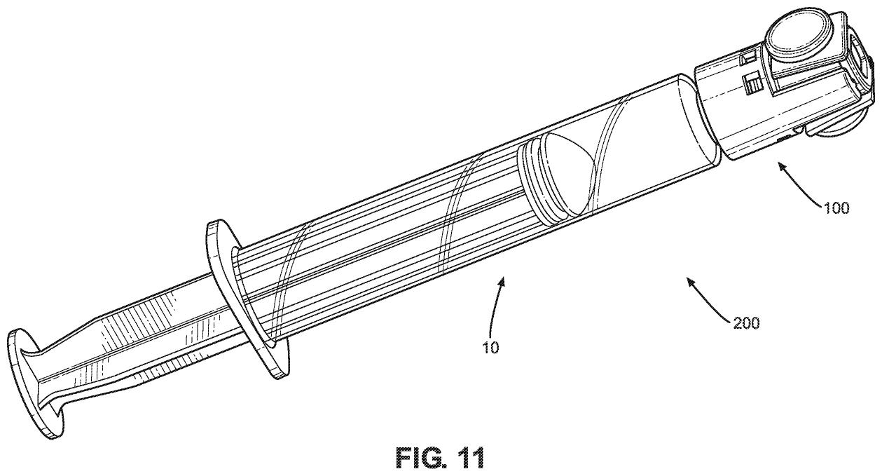

The closure assembly 100 may be used with various devices, for example medical devices. Suitable medical devices may include, but are not limited to, syringes, bottles, tubes, or other medical devices that may benefit from a closure assembly that indicates prior tampering with the assembly or the medical device. Referring to the illustrative embodiment of FIG. 11, the closure assembly 100 is fixedly attached to a syringe 10.

The closure assembly 100 may be manufactured, sold, and distributed as a stand-alone component that can be adapted to a desired use or device. Alternatively, the closure assembly 100 may be manufactured, sold, and distributed affixed to a desired device and intended for a specific use. Referring again to FIG. 11, the exemplary system 200 includes the closure assembly 100 fixedly attached to a syringe 10. The syringe 10 may be prefilled with a desired medicinal substance before distribution.

It will be understood that the closure assembly 100 may be attached to a desired device in a variety of ways, and this disclosure is not limited to a particular method of connecting the closure assembly 100 to a device. Referring once more to FIGS. 1-4, the cap 130 may be designed to communicate with the connector 104 in such a manner that the cap 130 and the connector 104 are rotated to engage the retaining mechanism 120 with a corresponding connector on the device. The connector 104 may include a ramp 124 that is configured to contact a corresponding ramp 134 on the cap 130. When the cap 130 is rotated in a first direction (e.g., clockwise around the axis A), the ramp 134 disposed on the cap 130 also moves and forcibly contacts the ramp 124. As force is exerted on the ramp 124 that is fixedly disposed on the connector 104, the connector 104 also rotates in the same first direction (e.g., clockwise), and the retaining mechanism 120 engages with the device. When the cap 130 is rotated in a second direction opposite the first direction (e.g., counterclockwise around the axis A), the ramp 134 contacts the ramp 124 and slides along it, such that even when the cap 130 rotates, the rotational force transferred to the ramp 124 is insufficient to rotate the connector 104.

In some embodiments, the cap 130 may have a first position relative to the connector 104 and a second position that is axially displaced from the first position. In the first position, the ramp 134 is at least partially located in the same plane as the ramp 124 such that the two ramps can contact each other upon rotation of the cap 130. In the second position, the cap 130 is axially displaced a sufficient distance such that the plane in which the ramp 134 lies no longer overlaps the plane of the ramp 124. When the cap 130 is in the second position, neither rotation in the first direction nor rotation in the second direction results in contact between the ramps 124, 134.

When the closure assembly 100 is in the locked configuration, rotation of the cap 130 around the connector 104 is limited or prevented. This allows the entire closure assembly 100 to be removed from the device. As the cap 130, the locking mechanism 150, and the connector 104 are “locked” together such that there is limited or no relative movement between the components, the closure assembly 100 may be twisted in a removing direction (e.g., counterclockwise) to disengage the retaining mechanism 120 of the connector 104 from the device or component to which it is connected. The closure assembly 100 can then be discarded.

The embodiments disclosed herein offer a number of advantages. The closure assembly 100 allows a user to quickly and accurately detect any tampering of the assembly or the device to which it is connected. In the medical field, this decreases the risk of inadvertently administering an incorrect medicinal substance to a patient. Furthermore, once the user moves the closure assembly 100 into the locked configuration, in some embodiments it cannot be moved back to the unlocked configuration. This serves to alert another user at a later time that the medical device with the locked closure assembly has already been used, thus decreasing risk of infection from reusing certain medical devices, for example, syringes.

The present disclosure offers various advantages over existing anti-tamper technology. Many current options require removal of a portion of the device to indicate tampering. For example, some existing assemblies require breaching or breaking a ring surrounding the assembly. This approach results in extraneous pieces of the assembly that need to be removed. In a medical scenario, loose pieces may not be desirable because they may cause contamination of tools and/or infection in the patient. Additionally, breaking or removing a portion of the assembly exposes sharp edges, the locations and angles of which are often difficult to predict. This may result in direct injury to the user and/or the patient, or in indirect risk of contamination or infection due to ripped gloves or the loose edges catching on tools or materials.

Furthermore, existing technology often requires application of greater force by the user. Some people, especially the young, the elderly, or those suffering from debilitating diseases, may not have the strength to operate these assemblies. The presently disclosed closure assembly is more ergonomic and requires less effort to toggle between the unlocked and the locked configuration. Moreover, the disclosed ergonomic assembly avoids excessive force and direct contact with dangerous portions of medical devices. Existing technology often requires direct access to the connecting interfaces of the devices, which may expose users to greater risk of injury, for example, contacting sharp edges, needles, or dangerous substances within the device.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

1. A tamper-evident closure assembly for use with a medical device,
the closure assembly comprising:
a connector having a receptacle defined by an outer surface of the connector, the receptacle having a depression and a wall adjacent the depression;
a cap configured to attach to the connector and to rotate around the connector; and
a locking mechanism disposed on the cap, the locking mechanism having a locked configuration and an unlocked configuration, the locking mechanism having a tab movable between the locked configuration and the unlocked configuration and a plug disposed on the tab,
wherein when the locking mechanism is in the unlocked configuration, the plug is disposed outside of the receptacle on the connector, and when the locking mechanism is in the locked configuration, the plug is disposed at least partly within the receptacle, such that the cap is prevented from rotating around the connector.

2. The tamper-evident closure assembly of claim 1, wherein the connector further includes a retaining mechanism configured to releasably affix the connector to a medical device.

3. The tamper-evident closure assembly of claim 2, wherein the medical device is a syringe.

4. The tamper-evident closure assembly of claim 1, wherein the locking mechanism further includes an actuator disposed on the tab, the actuator being configured to move the tab into the locked configuration from the unlocked configuration and into the unlocked configuration from the locked configuration.

5. The tamper-evident closure assembly of claim 4, wherein the actuator is a button configured to be depressed by a user.

6. (canceled)

7. (canceled)

8. The tamper-evident closure assembly of claim 1, wherein the connector includes a plurality of receptacles, and the locking mechanism includes a plurality of corresponding plugs configured to move into each of the plurality of receptacles when the locking mechanism is in the locked configuration.

9. The tamper-evident closure assembly of claim 1, wherein the cap further includes an opening disposed between the tab on the closure locking mechanism and the receptacle on the connector, the opening being sized such that the plug can be moved through the opening only in a first direction toward the receptacle and prevented from being moved through the opening in a second direction opposite the first direction.

10. The tamper-evident closure assembly claim 1, wherein the plug has a tapered distal end configured to slidingly contact the wall of the receptacle, such that when the closure mechanism is moved from an unlocked configuration to a locked configuration, the plug is slid along the wall into the receptacle.

11. (canceled)

12. (canceled)

13. The tamper-evident closure assembly of claim 1, further comprising an indicator configured to distinguish between the unlocked configuration and the locked configuration.

14. The tamper-evident closure assembly of claim 1, the connector further having a ramp and the cap further having a corresponding ramp configured to engage with the ramp on the connector such when the connector rotates in a first direction, the connector also rotates in the first direction.

15. A system for medicinal delivery, the system comprising:
a medical device configured to receive a medicinal substance; and
a tamper-evident closure assembly, the closure assembly having:
a connector having a retaining mechanism configured to releasably affix the connector to the medical device and a receptacle defined by an outer surface of the connector, the receptacle having a depression and a wall adjacent the depression;
a cap configured to attach to the connector and to rotate around the connector; and
a locking mechanism disposed on the cap, the locking mechanism having a locked configuration and an unlocked configuration, the locking mechanism having a tab movable between the locked configuration and the unlocked configuration and a plug disposed on the tab,
wherein when the locking mechanism is in the unlocked configuration, the plug is disposed outside of the receptacle on the connector, and when the locking mechanism is in the locked configuration, the plug is disposed at least partly within the receptacle, such that the cap is prevented from rotating around the connector.

16. The system of claim 15, wherein the medical device is a syringe.

17. (canceled)

18. The system of any of claim 15, wherein the locking mechanism further includes an actuator disposed on the tab, the actuator being configured to move the tab into the locked configuration from the unlocked configuration and into the unlocked configuration from the locked configuration.

19. The system of any of claim 15, wherein the actuator is a button configured to be depressed by a user.

20. (canceled)

21. (canceled)

22. The system of any of claim 15, wherein when the locking mechanism is in the locked configuration, the closure assembly can be twisted and disconnected from the medical device.

23. The system of claim 15, wherein the connector includes a plurality of receptacles, and the locking mechanism includes a plurality of corresponding plugs configured to move into each of the plurality of receptacles when the locking mechanism is in the locked configuration.

24. The system of claim 15, wherein the cap further includes an opening disposed between the tab on the locking mechanism and the receptacle on the connector, the opening being sized such that the plug can be moved through the opening only in a first direction toward the receptacle and prevented from being moved through the opening in a second direction opposite the first direction.

25. The system of claim 15, wherein the plug has a tapered distal end configured to slidingly contact the wall of the receptacle, such that when the closure mechanism is moved from an unlocked configuration to a locked configuration, the plug is slid along the wall into the receptacle.

26. (canceled)

27. (canceled)

28. The system of claim 15, further comprising an indicator configured to distinguish between the unlocked configuration and the locked configuration.

29. The system of claim 15, the connector further having a ramp and the cap further having a corresponding ramp configured to engage with the ramp on the connector such when the connector rotates in a first direction, the connector also rotates in the first direction.

30. A method of removing a tamper-evident closure assembly from a medical device, the method comprising:
moving the closure assembly from an unlocked configuration to a locked configuration, the closure assembly having a locking mechanism having a plug and a connector defining a receptacle thereon; and
rotating the closure assembly in the locked configuration until the closure assembly is disconnected from the medical device.

31. The method of claim 30, wherein moving the closure assembly into the locked configuration includes moving the plug into the receptacle.
